(19) United States
(12) Patent Application Publication
(10) Pub. No.: US 2009/0088620 A1
(43) Pub. Date: Apr. 2, 2009

(54) QUANTITATIVE CLINICAL AND PRE-CLINICAL IMAGING

(75) Inventors: Lyubomir ZAGORCHEV, Lebanon, NH (US); Douglas STANTON, Ossining, NY (US); Andrew BUCKLER, Wenham, MA (US); Yogish MALLYA, Bangalore (IN)

Correspondence Address: PHILIPS INTELLECTUAL PROPERTY & STANDARDS, 595 MINER ROAD, CLEVELAND, OH, 44143, US

(73) Assignee: KONINKLIJKE PHILIPS ELECTRONICS N. V., Eindhoven (NL)

(21) Appl. No.: 12/206,808

(22) Filed: Sep. 9, 2008

Related U.S. Application Data
(60) Provisional application No. 60/976,519, filed on Oct. 1, 2007.

(51) Int. Cl. A61B 5/05; G06G 7/60

(52) U.S. Cl. 600/407; 703/11

(57) ABSTRACT

In a clinical or preclinical imaging system, an image acquisition subsystem includes a data acquisition and image reconstruction elements generating clinical or preclinical images. A quantitative image processing subsystem generates variability metadata associated with the clinical or preclinical images, and a clinically or preclinically significant result with an associated confidence interval computed based on the variability metadata. A user interface displays the clinically or preclinically significant result together with the associated confidence interval. A phantom for calibrating such an imaging system includes a deformable nonbiological structure approximating structure of a clinical or preclinical subject to be imaged, and fiducial markers detectable by the imaging system disposed on or in the deformable nonbiological structure to move with deformation of the deformable nonbiological structure.

### CROSS REFERENCE TO RELATED APPLICATIONS

This application claims the benefit of U.S. provisional application Ser. No. 60/976,519 filed Oct. 1, 2007, which is incorporated by reference.

### BACKGROUND OF THE INVENTION

The following relates to the medical arts. The following finds illustrative application to clinical and pre-clinical imaging, and is described with particular reference thereto. However, the following will find application in other medical applications such as, but not limited to, measurement of diagnostically relevant parameters to aid in patient triage and management.

Medical therapies and diagnostic methods or systems in the research, development, and certification stages are typically tested pre-clinically on animals such as mice, guinea pigs, or so forth. If the pre-clinical tests are promising and indicate an acceptable level of safety, the development proceeds to clinical studies on human volunteers. Based on the results of such clinical studies, the efficacy and safety of the therapy or diagnostic method or system is determined, and commercial entities and relevant government regulatory agencies make decisions as to whether to authorize and proceed to use the therapy or diagnostic.

In pre-clinical and clinical studies, feedback in the form of medical imaging is sometimes solicited. For example, in a cancer treatment therapy, it may be desired to employ magnetic resonance (MR) imaging, computed tomography (CT) imaging, positron emission tomography (PET) imaging, x-ray imaging, or another imaging modality or combination of imaging modalities (i.e., multimodality imaging), to study the extent (if any) by which the therapy reduces the size, distribution, metabolic activity, or other anatomical or functional characteristics of the cancerous tumors.

A pre-clinical or clinical study is carefully designed by the responsible medical researchers, with considerable thought given to numerous design parameters including, for example, the number of animal or human volunteer subjects, the modality or modalities of medical imaging employed including a detailed understanding of the capabilities and limitations of each imaging modality such as resolution characteristics, level of sensitivity to various tissue types, impact of anesthesia, temperature, and other variables on the imaging, impact of subject motion on the imaging, and so forth. When multimodality imaging is employed, additional consideration is given to the effects of combining images from the various imaging modalities, such as errors introduced during spatial registration of images from different modalities.

Error, in the form of noise, lack of precision, and/or uncontrolled variation of whatever kind can be introduced at substantially any stage of the processing including during imaging data acquisition, image reconstruction, post-reconstruction image processing, multimodality spatial image registration, extraction of clinically significant results from reconstructed images, and so forth. Propagation of errors across the different stages can reduce confidence intervals indicative of the error, or in certain cases data fusion of, say, corroborating data derived from another source (e.g., a similar feature found in complementary input data such as images of different modalities or non-imaging data as complementary to imaging) can serve to increase confidence.

Ideally, the parameters impacting various error estimates, confidence intervals, and the error propagation are carefully determined and recorded by the researchers, so that the resulting pre-clinical or clinical study conclusions can be assessed in view of statistically significant error estimates. In practice, however, these estimates are typically performed manually using office-type spreadsheets such as Microsoft® Excel or other manually operated calculation aids, and in some such cases calculation of the resulting statistical significance with each form of error taken into account is either not done or itself subject to error. During image data acquisition, parameters relevant to making accurate error estimates and accurate error propagation estimates are sometimes not recorded, either inadvertently or because the study protocol did not foresee the need to record this information. Failure to determine, record, and preserve such information relevant to error or confidence interval assessment can lead to expensive and time-consuming pre-clinical or clinical studies that are fundamentally flawed and of limited or non-existent value for making informed decisions regarding the experimental therapy or diagnostic under investigation.

These problems are heightened when the study employs multimodality imaging. A prerequisite for synergistic comparison or combination of images from plural imaging modalities is accurate image processing, such as spatial registration of the images. Such spatial registration typically includes both rigid translational and/or rotational components, and elastic or deformational registration components. Both rigid and elastic or deformational registration algorithms are available. However, it is difficult or impossible using existing techniques to empirically determine the amount of error introduced by these registration operations. Accordingly, researchers typically assume that no error is introduced (which is almost certainly wrong) or make estimates of the introduced error based on first principles calculations or other non-empirical evidence. Existing calibration phantoms have substantial deficiencies and are not effective for calibrating, or assessing error introduced by, image registration techniques. In view of the increasingly common use of multimodality imaging in pre-clinical and clinical studies, this fundamental deficiency in image registration is problematic because it introduces error of largely unknown magnitude, nature, and effect on error propagation into the study analyses.

The following provides new and improved apparatuses and methods which overcome the above-referenced problems and others.

### SUMMARY OF THE INVENTION

In accordance with one aspect, a clinical or preclinical imaging method is disclosed, comprising: acquiring imaging data of clinical or preclinical subjects; reconstructing the imaging data to generate clinical or preclinical images; processing the clinical or preclinical images to generate a clinically or preclinically significant result; generating variability metadata respective to at least one of the acquiring, the reconstructing, and the processing; and estimating a confidence interval for the clinically or preclinically significant result based on the generated variability metadata.

In accordance with another aspect, a clinical or preclinical imaging system is disclosed, comprising: an image acquisition subsystem including a data acquisition element and an image reconstruction element cooperating to generate clinical or preclinical images of clinical or preclinical subjects; a quantitative image processing subsystem operating in cooperation with the image acquisition subsystem to generate (i) variability metadata associated with the clinical or preclinical images, (ii) a clinically or preclinically significant result, and (iii) a confidence interval associated with the clinically or preclinically significant result computed based on the variability metadata; and a user interface (60) configured to display the clinically or preclinically significant result together with the associated confidence interval.

In accordance with another aspect, a phantom is disclosed for calibrating a clinical or preclinical imaging system, the phantom comprising: a deformable nonbiological structure approximating structure of a clinical or preclinical subject to be imaged by the clinical or preclinical imaging system; and fiducial markers disposed on or in the deformable nonbiological structure so as to move with deformation of the deformable nonbiological structure, the fiducial markers being detectable by the clinical or preclinical imaging system.

In accordance with another aspect, a method of manufacturing a phantom simulating a biological subject is disclosed, the method comprising: forming a first deformable structure element using a selected material; curing the first deformable structure element using a first curing cycle to cause the first deformable structure element to have a first Hounsfield number approximating the Hounsfield number of a first tissue type; forming a second deformable structure element using the selected material; and curing the second deformable structure element using a second curing cycle different from the first curing cycle to cause the second deformable structure element to have a second Hounsfield number different from the first Hounsfield number and approximating the Hounsfield number of the second tissue type different from the first tissue type.

In accordance with another aspect, a clinical or preclinical workstation is disclosed, comprising a quantitative image processing subsystem configured to process clinical or preclinical images to generate a clinically or preclinically significant result, the quantitative image processing subsystem including a variability estimator that computes a confidence interval associated with the result based on variability factors and accounting for error propagation; and a user interface configured to display the clinically or preclinically significant result together with the associated confidence interval.

One advantage resides in enhanced value in clinical and preclinical studies due to automated generation of error and confidence interval information.

Another advantage resides in improved image registration.

Another advantage resides in improved efficiency in the design and implementation of clinical and preclinical studies.

Another advantage resides in the combination of in-vivo imaging data with in-vitro measurements, in-silico results, and/or ex-vivo histology to assess and/or improve statistical significant results.

Still further advantages of the present invention will be appreciated to those of ordinary skill in the art upon reading and understand the following detailed description.

### BRIEF DESCRIPTION OF THE DRAWINGS

The drawings are only for purposes of illustrating the preferred embodiments, and are not to be construed as limiting the invention.

FIG. 1 diagrammatically shows a system for performing clinical or preclinical imaging.

FIG. 2 diagrammatically shows a functional embodiment of the clinical or preclinical imaging system.

FIG. 3 diagrammatically shows a phantom for calibrating image registration or other aspects of clinical or preclinical imaging.

### DETAILED DESCRIPTION OF THE INVENTION

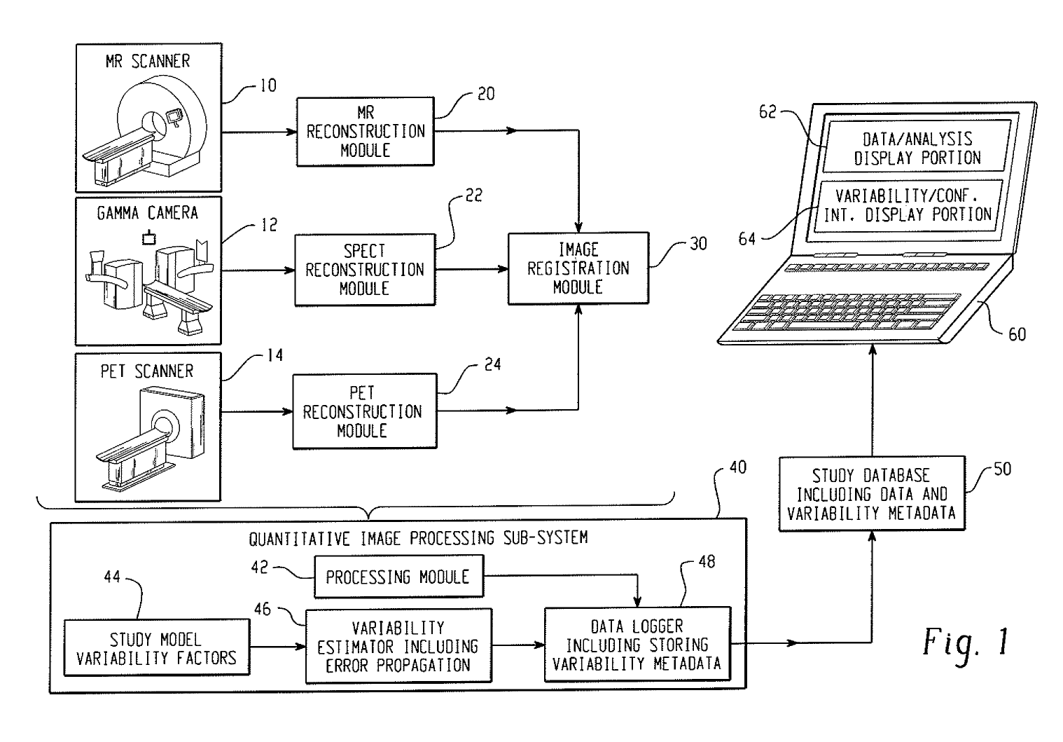

With reference to FIG. 1, a clinical or preclinical imaging system includes an image acquisition subsystem including a data acquisition element and an image reconstruction element cooperating to generate clinical or preclinical images of clinical or preclinical subjects. In illustrative FIG. 1, the clinical or preclinical imaging system includes three data acquisition elements, namely a magnetic resonance (MR) scanner 10, a gamma camera 12 for single photon emission computed tomography (SPECT) data acquisition, and a positron emission tomography (PET) scanner 14. The illustrative clinical or preclinical imaging system also includes three corresponding image reconstruction elements, namely an MR reconstruction module 20, a SPECT reconstruction module 22, and a PET reconstruction module 24. These illustrative components are examples; more generally, the clinical or preclinical imaging system can include as few as a single data acquisition element and corresponding reconstruction processor, or can include two data acquisition elements, the illustrated three data acquisition elements, or more data acquisition elements. A data acquisition element or elements can be provided to support substantially any imaging modality useful in clinical or preclinical studies, such as the illustrated MR, SPECT, or PET modalities, or computed tomography (CT), or fluoroscopy, or ultrasound, or so forth. Moreover, in some embodiments, the image reconstruction elements may be integrated with the data acquisition elements, or a single image reconstruction element may perform image reconstruction for data acquired by two or more different data acquisition elements. Similarly, although the illustrated three data acquisition elements 10, 12, 14 support different imaging modalities, it is also contemplated to have two or more data acquisition elements supporting the same imaging modality. Still further, a single data acquisition element may support two or more different imaging modalities, such as in the case of a combined PET/CT scanner. Such a data acquisition element that supports two or more different imaging modalities is sometimes referred to as a hybrid element. Some examples of suitable data acquisition elements for clinical imaging include the Achieva and Intera MR scanners supporting MR, the Brightview, Forte, and Skylight gamma cameras supporting SPECT, the Allegro, CPET, and Gemini scanners supporting PET, and the Precedence SPECT/CT hybrid scanner supporting both SPECT and CT imaging modalities, all of which are available from Koninklijke Philips Electronics N.V., Eindhoven, the Netherlands. Some examples of suitable data acquisition elements for preclinical imaging include any of the aforementioned elements for clinical imaging, as well as the Mosaic scanner supporting PET and specially designed for preclinical imaging, available from Koninklijke Philips Electronics N.V., Eindhoven, the Netherlands.

If the number of different imaging modalities supported by the image acquisition subsystem is greater than one, such as two different modalities, or the illustrated three different modalities, or more different modalities, then the image acquisition subsystem is a multimodal image acquisition subsystem, and the multimodal image acquisition subsystem optionally further includes a registration element 30 configured to spatially register images from different modalities. The registration element 30 receives input images from two or more different imaging modalities that are nominally of the same spatial region of the subject, and uses landmarks or other features to identify and spatially align features in the images to facilitate meaningful comparison or combination of the images acquired using the different modalities. The registration element 30 can implement one or more rigid registration techniques and/or one or more nonrigid registration techniques.

In order to support the clinical or preclinical study objectives, a quantitative image processing subsystem 40 operates in cooperation with the image acquisition subsystem to generate variability metadata associated with the clinical or preclinical images, one or more clinically or preclinically significant results, and a confidence interval associated with each clinically or preclinically significant result computed based on the variability metadata. A processing module 42 generates the one or more clinically or preclinically significant results. For example, the processing may in some embodiments include generation of a fused image combining images acquired by two or more different imaging modalities after spatial registration by the registration processor 30. In some embodiments, the processing may include segmentation of the images and characterization of a segmented region or regions of interest such as a tumor or plurality of tumor regions by one or more characterizing parameters such as size, tumor count, tumor area, tumor density (measured for example by Hounsfield units in a CT image), or so forth. In some embodiments, the processing may include generating a count of the number of clinical or preclinical subjects having a feature of interest, such as a tumor or other indicia of the presence of a pathology under study.

The processing module 42 produces results whose confidence interval is dependent upon the statistical variability of the underlying data acquisition, image reconstruction, and post-reconstruction processing operations. Study model variability factors 44 provide estimates for the statistical variability of each operation. For example, in the data acquisition operation some variability factors may include sensitivity, spatial resolution, energy resolution (in the case of imaging modalities such as SPECT and PET that employ energetic particle detectors), magnetic field homogeneity (in the case of MR), and so forth. Additional variability factors may be biological in origin, relating for example to temperature regulation of the subjects, anesthesia effects, subject motion blurring, species or individual subject variability, and so forth. Image reconstruction can also introduce variability such as known types of image artifacts, known approximations employed in the reconstruction, and so forth. The post-reconstruction processing can introduce still further variability, such as segmentation errors. The study model variability factors 44 provide quantitative information for each type or source of variability, derived empirically, based on first principles analysis, or so forth.

The confidence interval for a clinically or preclinically significant result or an intermediate result depends upon the variability of the preceding operations as well as the way in which such variability propagates from one operation to the next. A variability estimator 46 estimates the confidence intervals for each operation, taking into account error propagation across the preceding operations. Such error propagation can either magnify or reduce the extent of variability, depending upon the interaction of the succeeding operation respective to the preceding operation.

Advantageously, the quantitative image processing subsystem 40 integrates the study model variability factors 44 and variability estimator 46 into the clinical or preclinical imaging system, and the variability information is treated as metadata associated with the acquired data, reconstructed images, or other substantive data of the clinical or preclinical imaging system. For example, the data acquired by the data acquisition elements 10, 12, 14 are suitably tagged with relevant variability metadata such as imaging parameters (which collectively determine resolution or other variability), the reconstructed images output by the reconstruction processor 20, 22, 24 are suitably tagged with variability metadata such as computed resolution, subject temperature and temperature resolution, and so forth, and the clinically or preclinically significant results output by the processing module 42 are suitably tagged with variability metadata such as the resolution confidence interval for the determined tumor size. Because the quantitative image processing subsystem 40 including variability and confidence interval estimation elements 44, 46 are an integral part of the clinical or preclinical imaging system and operate automatically during imaging, it is ensured that information is generated that is sufficient to estimate confidence intervals for the clinically or preclinically significant results, so that the clinically or preclinically significant results are of diagnostic value.

Furthermore, a data logger 48 automatically logs the intermediate and final clinically or preclinically significant results along with the relevant variability metadata, for example stored as tags associated with the corresponding substantive information. As a result, a study database 50 stores the intermediate and final clinically or preclinically significant results and also stores the corresponding relevant variability metadata. In this way, reviewers or other retrospective analysts can review the study results, the corresponding confidence intervals, and the underlying sources of variability to ensure that the results are accurate, employed appropriate study protocols, and so forth.

It will be noted that the confidence interval estimation pathway 44, 46 is independent of the data acquisition, reconstruction, and processing elements 10, 12, 14, 20, 22, 24, 30, 42. As a result, if a retrospective analyst concludes that one of the underlying variability factors 44 was incorrect, or concludes that an error propagation transformation used in the variability estimator 46 was incorrect, the analyzt can readily correct this by inputting the corrected variability factor or transformation and re-applying the confidence interval estimation pathway 44, 46. The data logger 48 can either replace the metadata in the study database 50 with the corrected variability metadata, or can supplement the study database 50 with the corrected variability metadata, for example with a tag indicating date of correction and the identity of the person who performed the correction.

A user interface 60 display the study results. A data analysis/display portion 62 displays the substantive results, such as the reconstructed images and the fused images, tumor size and/or density parameters, or other clinically or preclinically significant results. A variability or confidence intervals display portion 64 displays the corresponding variability metadata. Although the display portions 62, 64 are shown as distinct regions of the display, more generally the display portions 62, 64 may be interleaved, superimposed, or otherwise combined. For example, the data analysis/display portion 62 may include a display of a reconstructed image, while the variability display portion 64 includes text superimposed on the displayed reconstructed image that provides resolution or other variability information.

In some embodiments, the user interface 60 also enables the analyst to input or modify the study model variability factors 44, adjust error propagation transformations applied by the variability estimator 46, input or adjust parameters used in data acquisition by the data acquisition elements 10, 12, 14, or otherwise control the clinical or preclinical imaging system.

In some embodiments, the confidence interval estimation pathway 44, 46 can be invoked prospectively, automatically, or retrospectively. For example, when researchers are designing the study they may prospectively (that is, prior to acquiring imaging data) invoke the confidence interval estimation pathway 44, 46 to determine the confidence intervals that will be achieved using the currently set parameters. If these confidence intervals are unsatisfactory, the researchers can adjust parameters such as the operational parameters of the data acquisition elements 10, 12, 14, the number of clinical or preclinical subjects, the subject preparations (such as whether to use anesthesia and if so how much), or so forth. The researchers would then again prospectively invoke the confidence interval estimation pathway 44, 46 to determine the effect on the confidence intervals of these adjustments. In this manner, the researchers can iteratively design the study protocol to achieve the desired confidence intervals prior to acquiring imaging data.

During the study, the confidence interval estimation pathway 44, 46 is optionally invoked automatically responsive to acquiring, reconstructing, and processing data to determine and log the variability metadata together with the substantive data (e.g., acquired data, reconstructed images, post-reconstruction generated clinically or preclinically significant results, or so forth). As noted previously, the confidence interval estimation pathway 44, 46 in some embodiments also can be invoked retrospectively to correct perceived errors in the underlying study model variability factors 44 and/or transformations used by the variability estimator 46 in determining error propagation.

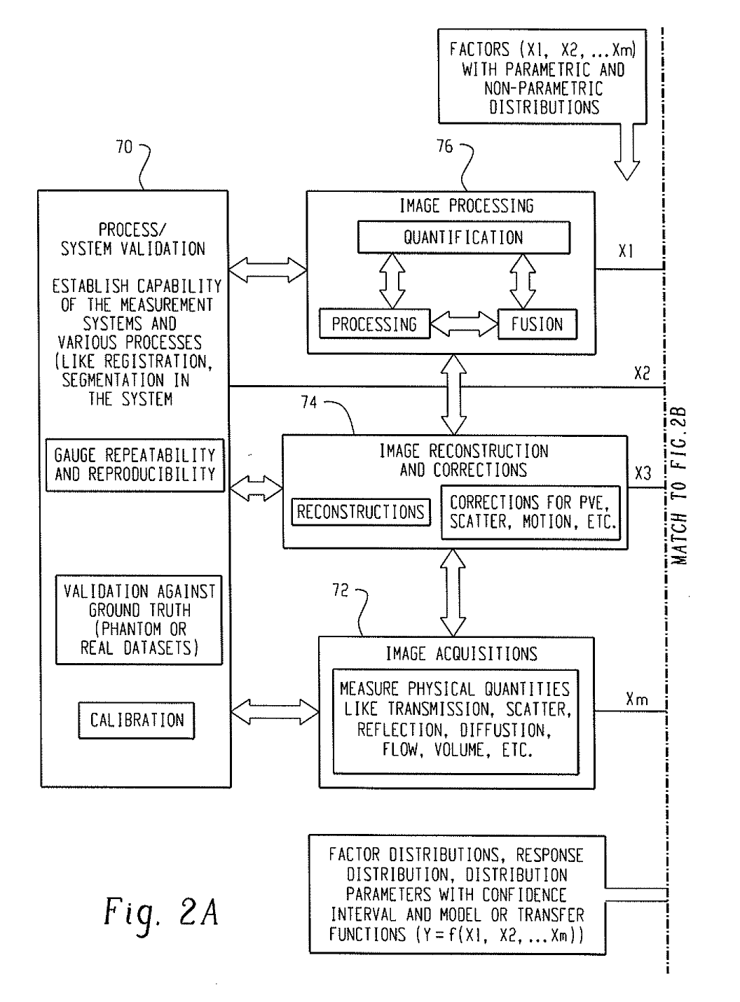

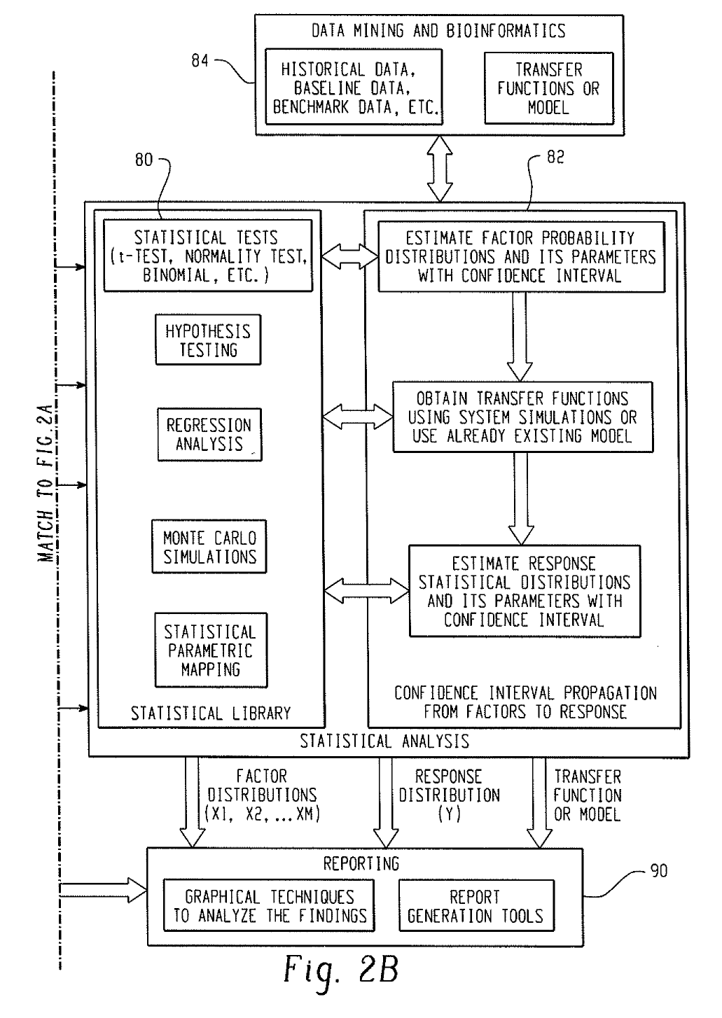

With reference to FIG. 2, an illustrative example of the confidence interval estimation pathway 44, 46 is described. Process/system validation operations 70 establish the capabilities of the data acquisition elements 10, 12, 14 and process components such as the reconstruction elements 20, 22, 24, registration module or element 30, and further processing modules or elements 42 such as segmentors. The process/system validation operations 70 perform operations such as gauging reproducibility and repeatability, validating against known parameters as defined by calibration phantoms or the like, and perform other calibration operations.

Image acquisition operations 72 measure physical quantities such as transmission, scatter, reflection, diffusion, flow, volume, and so forth. The specific physical quantities depend upon the implemented imaging modality. For example, the foregoing examples are useful for radiation-based imaging modalities, while additional or different parameters such as magnetic field homogeneity, gradient uniformity, and so forth are useful for MR modalities. Image reconstruction operations 74 performed by the reconstruction elements 20, 22, 24 are quantified by the confidence interval estimation pathway 44, 46 in terms of variability parameters such as corrections for PVE, scatter, motion, or so forth. Further processing operations 76 performed by the registration element 30 and processing module or modules 42 are similarly quantified by the confidence interval estimation pathway 44, 46 in terms of variability parameters such as estimated variability in the positions of the registered points of the image.

In performing the variability and confidence interval analyses, the confidence interval estimation pathway 44, 46 optionally utilizes a statistical library 80 containing various standard statistical functions such as statistical tests (t-test, normality test, binomial test, and so forth), hypothesis tests, regression analysis, Monte Carlo simulations, statistical parameter mapping, and so forth. To perform error propagation estimates 82, factor probability distributions and their parameters are estimated with confidence intervals, and are input to transfer functions or system simulations or pre-existing models of the various components of the clinical or preclinical imaging system, so as to generate response statistical distributions with propagated variability or confidence intervals. The error propagation estimates 82 also may utilize functions provided by the standard statistical library 80.

The operations of the confidence interval estimation pathway 44, 46 optionally also utilize data mining or bioinformatics 84, such as historical data, baseline data, benchmark data, and so forth, transfer functions or models developed based on past use of the clinical or preclinical imaging system, or so forth. The data mining or bioinformatics 84 are advantageously readily developed and maintained due to the tight integration of the confidence interval estimation pathway 44, 46 with the remainder of the clinical or preclinical imaging system.

The data output by the confidence interval estimation pathway 44, 46 are suitably reported in reporting operations 90 performed by the user interface 60, and may include for example graphical representations or interactive graphical analyses taking into account the confidence intervals, generation of reports for documenting the progress of the clinical or preclinical study, or so forth.

The functional arrangement set forth in FIG. 2 is an illustrative example. Other statistical functions, parameters, and so forth can be estimated or calculated, propagated through the data processing flow, and utilized. The functionality is suitably tailored to the specific imaging modality or modalities employed in the study and is suitably tailored to the goals of the study and the clinically or preclinically significant results to be obtained by the imaging. Post-processing steps of various kinds can be incorporated, for example including linear step-wise processing, and steps to combine data of various kinds, such as combination of imaging data from different imaging modalities using processes including spatial registration.

In addition to multimodality image fusion, it is also contemplated to combine or fuse imaging data with non-imaging data such as in-vitro measurements. For example, if non-imaging data is available that tends to show that a given subject has a pathology under study, then this non-imaging data can be taken into account to bias toward the conclusion or result that the given subject has the pathology under study. The non-imaging data is suitably also taken into account to adjust the confidence interval to reflect a higher confidence that the given subject has the pathology under study based on the available non-imaging data.

Various illustrative clinical or preclinical imaging systems and methods have been described with reference to FIGS. 1 and 2. In these systems and methods, the confidence interval estimation pathway 44, 46 makes use of variability parameters and error propagation transformations representative of the various system elements and operations. One of these is the registration module or element 30 used in the image processing 76 to estimate and propagate errors associated with the rigid or nonrigid image registration process. Such errors relate to mispositioning of points in the registered image.

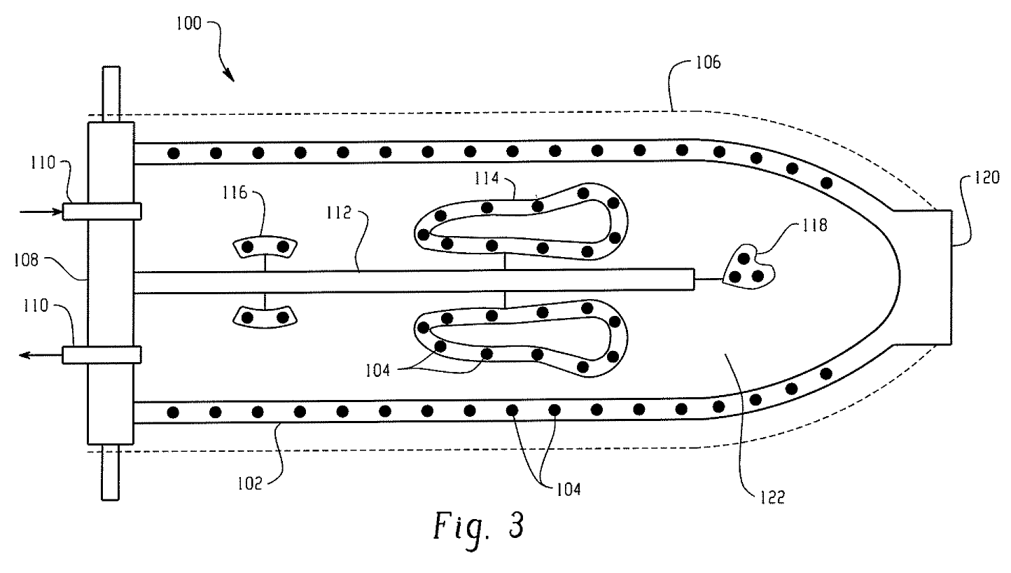

With reference to FIG. 3, in some embodiments a phantom 100 is provided to facilitate calibration of such image registration. The phantom 100 includes a deformable nonbiological structure 102 approximating structure of a clinical or preclinical subject to be imaged by the clinical or preclinical imaging system, and fiducial markers 104 disposed on or in the deformable nonbiological structure 100 so as to move with deformation of the deformable nonbiological structure. The fiducial markers 104 are selected to be detectable by the clinical or preclinical imaging system. If the clinical or preclinical imaging system is multimodal, then the fiducial markers 104 are preferably detectable by different modalities of the multimodal clinical or preclinical imaging system.

The illustrated phantom 100 has the deformable nonbiological structure 102 made of a vinyl or gel material such as polyvinyl alcohol (PVA), with the fiducial markers 104 formed as copper beads or other compact metal elements embedded in the PVA structure 102. In order to keep the PVA material moist, a hermetic sealant 106 surrounds the PVA-based deformable nonbiological structure 102. In the illustrated phantom 100, the hermetic sealant 106 is a container with an endcap 108 that seals one end and is optionally adapted for securing to a support structure of the data acquisition element, e.g. the MR scanner 10, gamma camera 12, or PET scanner 14. Optionally, openings 110 are provided to inject a contrast agent that is detectable by the imaging modality, so as to simulate contrast enhanced imaging. In the illustrated phantom 100, the deformable nonbiological structure 102 is mounted to a support post 112 that is in turn mounted to the endcap 108. Other mechanical support and sealant structures are also contemplated.

The deformable nonbiological structure 102 should approximate structure of a clinical or preclinical subject to be imaged by the clinical or preclinical imaging system, but the approximation does not need to be readily visually perceptible, and there can be substantial differences between the deformable nonbiological structure 102 and the structure of the subject that is being approximated. For example, the phantom 100 is suitable for approximating a rodent such as a mouse or rat, and the deformable nonbiological structure 102 has a generally cylindrical main section that approximates the main body of the mouse or rat and includes a lung structure 114, kidney structure 116, and heart structure 118 approximating the rodent's lungs, kidneys, and heart, respectively. Optionally, tubes may connect the openings 110 to a specific simulated organ, such as the heart structure 118, to enable simulation of injecting contrast agent into that organ. Similarly, the openings 110 could connect with the lung structure 114, which in such embodiments would be hollow, in order to simulate breathing.

The illustrated phantom 100 has, at the end of the container 106 distal from the endcap 108, an optional attachment point 120 for mounting that end of the phantom 100. Alternatively, the support can be single-ended utilizing only the endcap 108. An inner sealed volume 122 defined by the container 106 and endcap 108 is suitably filled a water, saline solution, or another fluid mimicking the mostly fluid composition of a living subject.

To provide accurate positioning information, the fiducial markers 104 are preferably rigid generally spherical elements. In some embodiments, the deformable nonbiological structure 102 comprises a plurality of vinyl or gel elements made of the same vinyl or gel material, such as PVA, but cured using different curing cycles such that the vinyl or gel elements have different Hounsfield numbers to mimic different types of tissues. In such a method of manufacturing a phantom, a first deformable structure element is formed using a selected material, with the fiducial markers 104 embedded in the structure element, and is cured using a first curing cycle to cause the first deformable structure element to have a first Hounsfield number approximating the Hounsfield number of a first tissue type. As an example, the first structure may be the lung structure 114 of the phantom 100. A second deformable structure element is formed using the same selected material, with the fiducial markers 104 embedded in the structure element, and is cured using a second curing cycle different from the first curing cycle to cause the second deformable structure element to have a second Hounsfield number different from the first Hounsfield number and approximating the Hounsfield number of the second tissue type different from the first tissue type. As an example, the second structure may be the kidney structure 116 of the phantom 100. This process can be continued to make the heart structure 120, the bulk structure 102, and so forth. Advantageously, by making routine changes in the curing time and/or temperature of a PVA material, a wide range of Hounsfield numbers approximating most common biological tissues can be achieved.

The illustrated phantom 100 is suitable for simulating a mouse, rat, or other small animal. However, the process is readily scaled up to larger subjects including full-scale human phantoms. The PVA or other vinyl or gel material is readily deformed to simulate various mechanical stresses on the subject, and as noted previously one can readily incorporate tubing to implement pneumatic or hydraulic cycling of the heart and/or lungs so as to simulate the cardiac and/or respiratory cycle.

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

Having thus described the preferred embodiments, the invention is now claimed to be:

1. A clinical or preclinical imaging method comprising:
acquiring imaging data of clinical or preclinical subjects;
reconstructing the imaging data to generate clinical or preclinical images;
processing the clinical or preclinical images to generate a clinically or preclinically significant result;
generating variability metadata respective to at least one of the acquiring, the reconstructing, and the processing; and
estimating a confidence interval for the clinically or preclinically significant result based on the generated variability metadata.

2. The clinical or preclinical imaging method as set forth in claim 1, wherein the generating of variability metadata includes generating acquisition variability metadata responsive to the acquiring.

3. The clinical or preclinical imaging method as set forth in claim 1, further comprising:
storing the variability metadata together with at least one of the imaging data, the clinical or preclinical images, and the clinically or preclinically significant result.

4. The clinical or preclinical imaging method as set forth in claim 1, wherein the processing comprises spatially registering clinical or preclinical images acquired using multimodality imaging, and the generating of variability metadata includes generating processing variability metadata respective to the spatial registering.

5. The clinical or preclinical imaging method as set forth in claim 4, further comprising:
acquiring calibration imaging data of a phantom including fiducial markers using said multimodality imaging; and
computing from the calibration imaging data one or more parameters used in the generating of variability metadata respective to the spatial registering.

6. The clinical or preclinical imaging method as set forth in claim 1, further comprising:
acquiring calibration imaging data of a phantom including fiducial markers using said multimodality imaging; and
computing from the calibration imaging data one or more parameters used in the generating of variability metadata.

7. The clinical or preclinical imaging method as set forth in claim 6, further comprising:
deforming the phantom to simulate deformation of a subject.

8. The clinical or preclinical imaging method as set forth in claim 1, wherein the generating of the variability metadata and the estimating of the confidence interval are performed prior to the acquiring of imaging data.

9. The clinical or preclinical imaging method as set forth in claim 8, further comprising:
iteratively adjusting parameters of the acquiring, the reconstructing, and the processing and repeating the generating after each iteration to optimize said parameters respective to the estimated confidence interval prior to the acquiring.

10. A clinical or preclinical imaging system comprising:
an image acquisition subsystem including a data acquisition element and an image reconstruction element cooperating to generate clinical or preclinical images of clinical or preclinical subjects;
a quantitative image processing subsystem operating in cooperation with the image acquisition subsystem to generate (i) variability metadata associated with the clinical or preclinical images, (ii) a clinically or preclinically significant result, and (iii) a confidence interval associated with the clinically or preclinically significant result computed based on the variability metadata; and
a user interface configured to display the clinically or preclinically significant result together with the associated confidence interval.

11. The clinical or preclinical imaging system as set forth in claim 10, further comprising:
a data storage configured to store (i) the clinical or preclinical images, (ii) configuration parameters of the image acquisition subsystem used in the acquisition of the clinical or preclinical images, and (iii) the variability metadata associated with the clinical or preclinical images.

12. The clinical or preclinical imaging system as set forth in claim 10, wherein:
the image acquisition subsystem is multimodal and further includes a registration element configured to spatially register images from different modalities; and
the quantitative image processing subsystem generates spatial registration variability metadata associated with the spatial registration.

13. The clinical or preclinical imaging system as set forth in claim 12, further comprising:
a phantom simulating a clinical or preclinical subject and including fiducial markers discernable by different modalities of the image acquisition subsystem, the quantitative image processing subsystem being configured to determine parameters for generating spatial registration variability metadata based on a calibration image of the phantom generated by the image acquisition subsystem.

14. The clinical or preclinical imaging system as set forth in claim 10, wherein the variability metadata is generated by the quantitative image processing subsystem respective to at least one of (i) image acquisition by the data acquisition element, (ii) image reconstruction by the image reconstruction element, and (iii) processing by the quantitative image processing subsystem that generates the clinically or preclinically significant result.

15. The clinical or preclinical imaging system as set forth in claim 10, wherein the quantitative image processing subsystem generates the clinically or preclinically significant result by fusing at least some non-imaging data with the clinical or preclinical images, and the computation of the confidence interval associated with the clinically or preclinically significant result takes into account the fusing of the non-imaging data.

16. A phantom for calibrating a clinical or preclinical imaging system, the phantom comprising:
a deformable nonbiological structure approximating structure of a clinical or preclinical subject to be imaged by the clinical or preclinical imaging system; and
fiducial markers disposed on or in the deformable nonbiological structure so as to move with deformation of the deformable nonbiological structure, the fiducial markers being detectable by the clinical or preclinical imaging system.

17. The phantom as set forth in claim 16, wherein the clinical or preclinical imaging system is multimodal and the fiducial markers are detectable by different modalities of the multimodal clinical or preclinical imaging system.

18. The phantom as set forth in claim 16, wherein the deformable nonbiological structure comprises a vinyl or gel structure.

19. The phantom as set forth in claim 16, wherein the deformable nonbiological structure comprises a plurality of vinyl or gel elements made of the same vinyl or gel material but cured using different curing cycles such that the vinyl or gel elements have different Hounsfield numbers to mimic different types of tissues.

20. The phantom as set forth in claim 16, further comprising:
inflow openings configured for injection of a contrast agent into the deformable nonbiological structure so as to simulate contrast enhanced imaging.

21. A method of manufacturing a phantom simulating a biological subject, the method comprising:
forming a first deformable structure element using a selected material;
curing the first deformable structure element using a first curing cycle to cause the first deformable structure element to have a first Hounsfield number approximating the Hounsfield number of a first tissue type;
forming a second deformable structure element using the selected material; and
curing the second deformable structure element using a second curing cycle different from the first curing cycle to cause the second deformable structure element to have a second Hounsfield number different from the first Hounsfield number and approximating the Hounsfield number of the second tissue type different from the first tissue type.

22. The method as set forth in claim 21, wherein the selected material is a polyvinyl alcohol (PVA) material.

23. The method as set forth in claim 21, further comprising:
disposing fiducial markers on or in at least one of the first deformable structure element and the second deformable structure element such that the disposed fiducial markers move with deformation.

24. A clinical or preclinical workstation comprising:
a quantitative image processing subsystem configured to process clinical or preclinical images to generate a clinically or preclinically significant result, the quantitative image processing subsystem including a variability estimator that computes a confidence interval associated with the result based on variability factors and accounting for error propagation; and
a user interface configured to display the clinically or preclinically significant result together with the associated confidence interval.

25. The clinical or preclinical workstation as set forth in claim 24, further comprising:
an image registration module configured to spatially register clinical or preclinical images acquired using different imaging modalities, the variability estimator computing the confidence interval based in part on variability factors for the registration determined from images of a phantom including fiducial markers acquired using the different imaging modalities.
